(19) United States
(12) Patent Application Publication
(10) Pub. No.: US 2008/0286304 A1
(43) Pub. Date: Nov. 20, 2008

(54) Modifying Leukocyte Function

(75) Inventor: Ian Stuart Pardoe, Birmingham (GB)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HENDERSON MORLEY PLC, Moseley, Birmingham, W. Midlands (GB)

(21) Appl. No.: 12/067,406

(22) PCT Filed: Sep. 25, 2006
(86) PCT No.: PCT/GB2006/003554

§ 371 Date: Mar. 19, 2008

(30) Foreign Application Priority Data
Sep. 24, 2005 (GB) 0519554.0

(51) Int. Cl. A61K 39/12; C12N 5/06

(52) U.S. Cl. 424/204.1; 435/375

(57) ABSTRACT

Use of PREPS and/or L-particles for the manufacture of a medicament for modifying or regulating leukocyte function.

This invention relates to modifying leukocyte function, for example lymphocyte function, specifically, although not exclusively, cytotoxic T lymphocyte (CTL) function.

Cytotoxic T Lymphocytes (CTLs) play a vital role in controlling a variety of human illnesses including a wide variety of acute and chronic viral infections such as herpes simplex virus infection. CTLs are also thought to play a role in cancer surveillance i.e. preventing the spread of cancer in a patient already subject to disease, and also preventing sporadic cancer cells from developing into tumor masses. However, CTLs can also cause life-threatening illnesses such as transplant rejection, graft-vs host disease, and autoimmunity. Thus, CTLs play an important role in not only preventing disease but also in the manifestation of many diseases.

Clearly the ability to specifically modulate CTL function would be beneficial in the treatment of many conditions.

CTLs are a critical component of many successful immune responses, but they have the potential to injure the host. Therefore, their activity is closely regulated by multiple receptor-ligand interactions. Before they can function as effectors, CTLs must be activated by Antigen Presenting Cells (APC) and triggered by target cells. Once activated, the immune system has mechanisms to control the CTL response and prevent the destruction of healthy host cells. Alternatively, activated CTLs can become anergic or differentiate into a functionally impaired, non-responsive, or memory-like state.

It is one object of this invention to provide means to regulate or modify CTL activity.

A first aspect of the invention provides a method of treating a disease which is exemplified by over activity or dysfunction of leukocyte (e.g. CTL) function, the method comprising regulating or modifying leukocyte function by exposing leukocytes to one or both of PREPS and/or L-particles.

A second aspect of the invention provides a method of modifying or regulating leukocyte function, the method comprising exposing leukocytes (e.g. CTLs) to one or both of PREPS and/or L-particles.

A third aspect of the invention provides use of PREPS and/or L-particles for the manufacture of a medicament for modifying or regulating leukocyte (e.g. CTL) function.

A fourth aspect of the invention provides use of PREPS and/or L-particles for the manufacture of a medicament for the treatment of a condition or condition exemplified by over activity or dysfunction of leukocyte (e.g. CTL) function.

The disease or condition may include one or more of
Graft versus host disease
Acute organ rejection
Hashimotos thyroiditis,
Primary Myxoedema
Thyrotoxicosis
Pernicious anaemia
Addisons disease
Insulin dependant diabetes mellitus
Goodpastures syndrome
Pemphigus vulgaris
Pemphigoid
Sympathetic ophthalmia
Autoimmune haemolytic anaemia
Idiopathic thrombocytopaenic purpura
Idiopathic leucopenia
Primary biliary cirrhosis
Active chronic hepatitis
Ulcerative colitis
Sjogrens syndrome
Rheumatoid arthritis
Dermatomyositis
Scleroderma
Discoid lupus
Systemic lupus erythematosus

The PREPS and/or L-particles may be used as an immunotherapy and/or vaccine.

Excipients and/or carriers may be included. Adjuvants may be included in any medicament, such as, for example, aluminium salts, MPL and RC-529 (both supplied by Corixia Corporation). The medicament may be configured for topical, local or systemic administration.

U.S. Pat. No. 5,384,122 (assigned to the current applicant) discloses non-infectious particles called L-particles which are produced by herpesvirus-infected cells. The L-particles consist of tegument surrounded by envelope but lack the viral capsid and DNA and, consequently, are non-infectious.

U.S. Pat. No. 5,384,122 (the entire disclosure of which is incorporated by reference herein) discloses that L-particles may be used as an active component as a vaccine for use in the treatment of herpesvirus. Moreover, using recombinant herpesvirus containing DNA encoding a foreign protein or peptide the L-particles can be prepared to contain the foreign protein or peptide thereby enabling such L-particles to be used in combatting infectious diseases other than herpesvirus infections.

U.S. Pat. No. 5,994,116 (assigned to the current applicant) discloses a further type of herpes virus particle designated pre-viral DNA replication enveloped particles (PREPS). PREPS lack viral capsid and DNA and they contain reduced amounts of certain proteins, as compared to L-particles and increased amounts of other proteins, as compared to L-particles.

U.S. Pat. No. 5,994,116 (the entire disclosure of which is incorporated by reference herein) discloses that PREPS may be used as an active component of a vaccine for use in the treatment of a vaccine. It is also disclosed that recombinant techniques may be used to produce PREPS which have foreign proteins or peptides incorporated therein, thereby enabling PREPS to be used to combat infectious diseases other than herpes viruses.

Because CTLs are involved in controlling the acute, lytic phase and persistent, latent phase of herpes simples virus (HSV) infection, HSV has evolved numerous mechanisms to evade CTL as part of its survival strategy.

In a study by Sloan et al, (J. Immunol. (2003) 171 pp 6733-6741) it has been demonstrated that HSV-infected fibroblasts transmit a functionally inhibiting signal to CTL without infecting CTL or inducing apoptosis in CTL. These workers refer to this process as inactivation. The inactivating signal markedly decreases the cytotoxic and cytokine effector functions of CTL, and the inactivated phenotype is sustainable after CTL are removed from HSV-infected cells and treated with IL-2.

It is proposed that the inactivation of CTLs occurs due to the effect of L-particles and PREPS particles on CTL function, and it is the exploitation of this property that leads to the use of PREPS particles and L-particles as immunotherapeutic tools for the treatment of diseases of CTL function.

PREPS and L-particles can be made in accordance with the above-identified patents

Specific use of α-herpesvirus-derived particles as treatments for CTL mediated disease are as follows:

### Prevention of Acute Rejection of Corneal Transplant

It has been known for over 15 years (Opthalmol. 1989 96(1) pp 38-44) that the risk of rejection of a corneal transplant in man is greatest when the concentration of white cells in the graft bed is high. The rejection of the cornea is mediated by CTL and hence the application of L-particles or PREPS particles to either the corneal bed or to the newly transplanted cornea—either by topical application or by means of a bolus injection or delivery device, will lead to inactivation of these CTL and therefore reduce the risk of acute organ rejection.

### Prevention of Acute Rejection of Renal Transplantation

A longstanding study examining renal transplants in non human primates (who received poorly matched grafts) has shown that graft survival may be increased by inhibiting immune function immediately prior to surgery. This study indicates that the innate immune system is poised to defeat allograft tolerance induction, so effective blockade of innate immunity must be in place early, to enable development of a tolerogenic environment. (Transpl. Immunol. 2003 11(3-4): pp 335-44.

It is therefore feasible to administer PREPS or L-particles by systemic injection (intravenous or sub cutaneous) or by local injection to the site of lymph nodes of the drainage area of transplant (for example inguinal nodes if intra-abdominal transplantation was planned) or to any suitable site, before (e.g. a few days prior to) surgery to reduce the risk of immediate transplant rejection.

### Injection of Joints Affected by Acute Rheumatoid Arthritis

Rheumatoid arthritis is a condition characterised by severely inflamed joints which become both painful dysfunctional and which become structurally altered by the disease.

Recent research (Clin. Exp. Rheumatol. 2005; 23(2): pp 185-92) has demonstrated that joints that are heavily infiltrated by CTL are more likely to lead to disease progression with loss of normal structure. Application of PREPS or L particles by direct intra-articular injection leads to inhibition of CTL responses and therefore a reduction of the inflammatory and tissue destructive responses.

### Prevention of Systemic Diseases Caused by Overactive or Dysfunctional CTL

This includes a large number of conditions labelled as “connective tissue disorders” including dermatomyositis, scleroderma, SLE etc. and acute or chronic virus infections such as hepatitis C and hepatitis B. Delivery of PREPS or L particles to the haemopoetic system (bone marrow/liver/spleen/thymus) is useful method in the modulation of CTL function.

### Development of Specific Immuno-Modulating Preps or L-Particles

While the invention thus far described relates to L-particles or PREPS wholly composed of elements of herpes virus, a wider range of protection could be provided by producing recombinant herpes viruses expressing inserted foreign DNA in such a way as to incorporate the resulting foreign proteins or epitopic peptides from unrelated viruses or other types of herpes virus or other organisms into the L-particles or PREPS. (The term “foreign” herein means not native to the strain of herpes virus from which the L-particles or PREPS are derived).

Where the foreign DNA is foreign only in the sense that it is derived from another strain or type of HSV, it is expected that a recombinant HSV containing the foreign DNA will express it without difficulty in most cases, so that the protein thus produced becomes incorporated within the L-particles or PREPS (e.g. in the envelope or in the tegument). Recombinant virus expressing the foreign protein can be constructed by inserting the gene for that protein placed under the control of appropriate HSV signals in the genome of either the wild type HSV-1 virus or the DNA-replication negative HSV-1 using standard techniques (Rixon and McLauchlan, 1993, In: Molecular Virology, A Practical Approach, p. 285-307; ed. Davison A. J. and Elliott, R. M. IRL Press, Oxford). The wild type virus carrying the foreign gene can either be used with DNA-replication inhibitors (see above) or can be engineered further to render it DNA-replication negative. PREPS or L-particles containing foreign proteins could also be produced by treating cells engineered to express the foreign genes, carrying appropriate herpes virus signals, with the DNA-replication negative HSV-1 or with wild type HSV in the presence of DNA replication inhibitors.

Cytotoxic T lymphocytes (CTLs) are capable of conferring protection against intracellular pathogens and tumor. Protective antiviral immunity, mediated by the activation of antigenic epitope-specific CTL, can be achieved by delivering exogenous antigen into the cytosol of antigen-presenting cells. Cytosolic introduction of vaccine antigen, however, requires a specialized delivery strategy due to the membrane barrier limiting the access of macromolecules to the cytosol; L-particles and PREPS offer such a technique. The importance of using such techniques is that specific T cell responses may be affected by the delivery of non-viral proteins or antigens to T cells, which would modify the cellular immune function in response to such an antigen or antigens—including the specific down regulation of T cell responses by the use of appropriate proteins/antigens.

Whilst the above description details the modification of CTL function, it is within the scope of this invention to use PREPS and/or L-particles to modify the function of other lymphocytes (e.g. B cells, natural killer cells) and, indeed, preferably all or at least some leukocytes in the animal blood system.

1. A medicament for modifying or regulating leukocyte function, said medicament comprising at least one of PREPS and L-particles in an amount effective to modify or regulate the leukocyte function.

2. The medicament according to claim 1, wherein the leukocyte function is T lymphocyte function.

3. A medicament for treating a condition exemplified by over activity or dysfunction of CTL function, said medicament comprising at least one of PREPS and L-particles in an amount effective to modify or regulate the CTL function.

4. The medicament according to claim 3, wherein the condition is a member selected from the group consisting of:
Graft versus host disease,
Acute organ rejection,
Hashimotos thyroiditis,
Primary Myxoedema,
Thyrotoxicosis,
Pernicious anaemia,
Addisons disease,
Insulin dependant diabetes mellitus,
Goodpastures syndrome,
Pemphigus vulgaris,
Pemphigoid,
Sympathetic ophthalmia,
Autoimmune haemolytic anaemia,
Idiopathic thrombocytopaenic purpura,
Idiopathic leucopenia,
Primary biliary cirrhosis,
Active chronic hepatitis,
Ulcerative colitis,
Sjogrens syndrome,
Rheumatoid arthritis,
Dermatomyositis,
Scleroderma,
Discoid lupus, and
Systemic lupus erythematosus.

5. The medicament according to claim 1, in a form of an immunotherapy and/or a vaccine.

6. The medicament according to claim 1, further comprising at least one excipient, carrier and adjuvant.

7. The medicament according to claim 1, configured for topical, local or systemic administration.

8. A method of treating a disease which is exemplified by over activity or dysfunction of leukocyte function, the method comprising regulating or modifying leukocyte function by exposing leukocytes to at least one of PREPS and L-particles.

9. The method according to claim 8, wherein the disease is a member selected from the group consisting of:
Graft versus host disease,
Acute organ rejection,
Hashimotos thyroiditis,
Primary Myxoedema,
Thyrotoxicosis,
Pernicious anaemia,
Addisons disease,
Insulin dependant diabetes mellitus,
Goodpastures syndrome,
Pemphigus vulgaris,
Pemphigoid,
Sympathetic ophthalmia,
Autoimmune haemolytic anaemia,
Idiopathic thrombocytopaenic purpura,
Idiopathic leucopenia,
Primary biliary cirrhosis,
Active chronic hepatitis,
Ulcerative colitis,
Sjogrens syndrome,
Rheumatoid arthritis,
Dermatomyositis,
Scleroderma,
Discoid lupus, and
Systemic lupus erythematosus.

10. A method of modifying or regulating leukocyte function, the method comprising exposing leukocytes to at least one of PREPS and L-particles.
